(19) United States
(12) Patent Application Publication
(10) Pub. No.: US 2023/0410001 A1
(43) Pub. Date: Dec. 21, 2023

(54) SCHEDULE MANAGEMENT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Tokyo-to (JP); Toru MIYAGAWA, Seto-shi (JP); Hideo HASEGAWA, Nagoya-shi (JP); Yusuke KINOSHITA, Tokyo-to (JP); Hiroshi SHIMADA, Tajimi-shi (JP)

(73) Assignee: TOYOTA JIDOSHA KABUSHIKI KAISHA, Toyota-shi (JP)

(21) Appl. No.: 18/142,352

(22) Filed: May 2, 2023

(30) Foreign Application Priority Data
Jun. 21, 2022 (JP) 2022-099321

(51) Int. Cl. G06Q 10/0631

(52) U.S. Cl. CPC G06Q 10/063114

(57) ABSTRACT

A schedule management system includes: an acquisition unit configured to acquire information about a target exercise amount of a first user for each predetermined period; a place assignment unit configured to extract at least one working place from a database and assign the extracted at least one working place to the first user as a working place for the predetermined period, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered; and an output unit configured to output information about the working place assigned to the first user.

### CROSS REFERENCE TO RELATED APPLICATIONS

This application is based upon and claims the benefit of priority from Japanese patent application No. 2022-099321, filed on Jun. 21, 2022, the disclosure of which is incorporated herein in its entirety by reference.

### BACKGROUND

The present disclosure relates to a schedule management system, its control method, and a control program.

In recent years, it has been desired, for example, for workers in workplaces, to achieve both the performance of tasks assigned to them and exercises for maintaining their good health. For example, Japanese Unexamined Patent Application Publication No. 2018-84911 discloses a related technology.

Japanese Unexamined Patent Application Publication No. 2018-84911 discloses a worker information detection system that determines that a worker is in a warning state when a cumulative load determined from load information of the worker exceeds a threshold.

### SUMMARY

However, the system disclosed in Japanese Unexamined Patent Application Publication No. 2018-84911 merely issues a warning when there is a possibility that a worker (a user) is forced to work too much, and does not assign, to the worker, a working place with which the worker can achieve an appropriate amount of exercise (hereinafter also referred to as an “exercise amount”). Therefore, there is a problem in the system disclosed in Japanese Unexamined Patent Application Publication No. 2018-84911 that it is impossible to propose a work plan by which a worker can achieve both the performance of a task assigned to him/her and an exercise for maintaining his/her good health.

The present disclosure has been made in view of the above-described problem, and an object thereof is to provide a schedule management system, its control method, and a control program capable of proposing a work plan by which a user can achieve both the performance of a task assigned to him/her and an exercise for maintaining his/her good health.

A schedule management system according to the present disclosure includes: an acquisition unit configured to acquire information about a target exercise amount of a first user for each predetermined period; a place assignment unit configured to extract at least one working place from a database and assign the extracted at least one working place to the first user as a working place for the predetermined period, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered; and an output unit configured to output information about the working place assigned to the first user by the place assignment unit. The above-described schedule management system can assign, to the user, a working place with which the user can achieve an appropriate exercise amount. That is, the above-described schedule management system can propose a work plan by which a user can achieve both the performance of a task assigned to him/her and an exercise for maintaining his/her good health.

The exercise amount for each of the plurality of predetermined working places that is achieved when the first user moves from the residence place of the first user to the working place may be determined based on a traveling environment from the residence place to the working place.

The schedule management system may further include an exercise amount calculation unit, in which the acquisition unit may further acquire information about the residence place of the first user and the plurality of predetermined working places, and the exercise amount calculation unit may calculate, for each of the plurality of predetermined working places, an exercise amount that is achieved when the first user moves from the residence place of the first user to that working place based on the information about the residence place and the plurality of predetermined working places acquired by the acquisition unit, and register the calculated exercise amounts in the database.

The exercise amount for each of the plurality of predetermined working places that is achieved when the first user moves from the residence place to the working place may include an exercise amount during a work performed in the working place.

The acquisition unit may further acquire information about a first working place where the first user should stay for a first period included in the predetermined period, and the place assignment unit may extract, from the database, at least one working place which includes the first working place for the first period and with which the first user achieves the exercise amount corresponding to his/her target exercise amount for each predetermined period, and assign the extracted at least one working place to the first user as the working place for the predetermined period.

The acquisition unit may further acquire information about a second working place to which the first user has determined that he/she cannot move, and the place assignment unit may extract, from the database, at least one working place which is different from the second working place and with which the first user achieves the exercise amount corresponding to his/her target exercise amount for each predetermined period, and assign the extracted at least one working place to the first user as the working place for the predetermined period.

The acquisition unit may acquire information about a congestion state of each of the plurality of predetermined working places, and the place assignment unit may extract, from the database and based on the congestion states, at least one working place with which the first user achieves the exercise amount corresponding to his/her target exercise amount for each predetermined period, and assign the extracted at least one working place to the first user as the working place for the predetermined period.

The acquisition unit may acquire, as feedback information, information about an actual exercise amount performed by the first user as he/she has moved to the working place assigned to the first user, and register the acquired actual exercise amount in the database.

The information about the actual exercise amount performed by the first user may be at least one of a heart rate, a pulse rate, the number of steps, a calorie consumption of the first user acquired by a wearable terminal attached to the first user, and an amount of change in a weight of the first user measured by a scale.

The target exercise amount may be a target calorie consumption of the first user.

The target exercise amount may be determined based on a difference between a target weight and a current weight of the first user.

The target exercise amount may be determined based on a difference between a target body fat percentage and a current body fat percentage of the first user.

The target exercise amount may be determined based on a difference between a target body shape and a current body shape of the first user.

The output unit may display information about the working place assigned to the first user on a monitor.

The acquisition unit may acquire, for each of a plurality of users including the first user, information about the target exercise amount for each predetermined period; the place assignment unit may extract, from the database, for each of the plurality of users, at least one working place with which that user achieves an exercise amount corresponding to his/her target exercise amount for each predetermined period, and assign the extracted at least one working place to that user as the working place for the predetermined period; and the output unit may output information about the working places assigned to the users by the place assignment unit.

A method for controlling a schedule management system according to the present disclosure includes: acquiring information about a target exercise amount of a first user for each predetermined period; extracting at least one working place from a database, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered; assigning the at least one working place extracted from the database to the first user as a working place for the predetermined period; and outputting information about the working place assigned to the first user. By the above-described method for controlling a schedule management system, it is possible to assign, to the user, a working place with which the user can achieve an appropriate exercise amount. That is, by the above-described method for controlling a schedule management system, it is possible to propose a work plan by which a user can achieve both the performance of a task assigned to him/her and an exercise for maintaining his/her good health.

A control program according to the present disclosure causes a computer to perform: a process for acquiring information about a target exercise amount of a first user for each predetermined period; a process for extracting at least one working place from a database, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered; a process for assigning the at least one working place extracted from the database to the first user as a working place for the predetermined period; and a process for outputting information about the working place assigned to the first user. By the program, it is possible to assign, to the user, a working place with which the user can achieve an appropriate exercise amount. That is, by the above-described program, it is possible to propose a work plan by which a user can achieve both the performance of a task assigned to him/her and an exercise for maintaining his/her good health.

According to the present disclosure, it is possible to provide a schedule management system, its control method, and a control program capable of proposing a work plan by which a user can achieve both the performance of a task assigned to him/her and an exercise for maintaining his/her good health.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a schedule management system according to a first embodiment;

FIG. 2 is a block diagram showing an example of a configuration of a schedule management apparatus provided in the schedule management system shown in FIG. 1;

FIG. 3 is a flowchart showing operations performed by the schedule management system shown in FIG. 1;

FIG. 4 is a conceptual diagram showing an example of, for each of a plurality of predetermined working places, an exercise amount that is achieved as a user moves from his/her residence place to that working place, registered in a database;

FIG. 5 is a conceptual diagram for explaining an example of operations performed by the schedule management system shown in FIG. 1;

FIG. 6 is a conceptual diagram for explaining another example of operations performed by the schedule management system shown in FIG. 1;

FIG. 7 is a block diagram showing a modified example of the schedule management system shown in FIG. 1;

FIG. 8 is a block diagram showing an example of a configuration of a schedule management apparatus provided in the schedule management system shown in FIG. 7;

FIG. 9 is a block diagram showing an example of a configuration of a schedule management system according to a second embodiment;

FIG. 10 is a conceptual diagram for explaining an example of operations performed by the schedule management system shown in FIG. 9;

FIG. 11 is a conceptual diagram showing an example of, for each of a plurality of predetermined working places, an exercise amount that is achieved as a user moves from his/her residence place to that working place, registered in a database; and

FIG. 12 is a conceptual diagram for explaining another example of operations performed by the schedule management system shown in FIG. 9.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

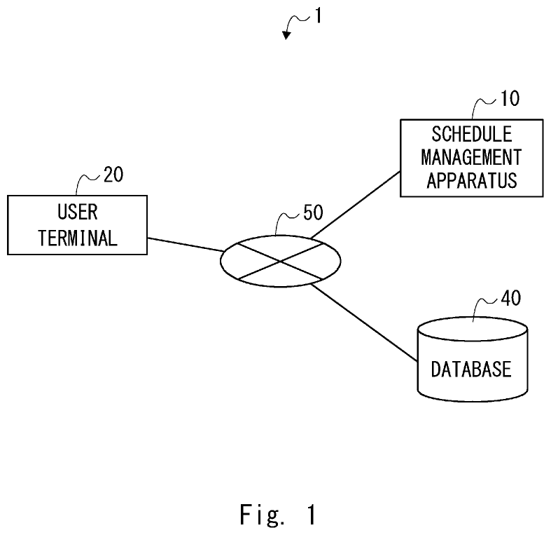

FIG. 1 is a block diagram showing an example of a configuration of a schedule management system 1 according to a first embodiment. The schedule management system 1 extracts at least one working place with which a user achieves an amount of exercise (hereinafter also referred to as an “exercise amount”) corresponding to his/her target exercise amount for each predetermined period (e.g., at least one working place with which a user achieves an exercise amount corresponding to his/her target exercise amount for each predetermined period by visiting there from his/her house and/or returning from there to his/her house) from a database in which information about, for each of a plurality of predetermined working places, an exercise amount that is achieved when the user moves from his/her residence place to that working place is registered, and assigns the extracted at least one working place to the user as a working place for the predetermined period (for each predetermined period, i.e., for a respective predetermined period). In this way, the schedule management system 1 can assign, to the user, a working place with which the user can achieve an appropriate exercise amount. That is, the schedule management system 1 can propose a work plan by which a user can achieve both the performance of a task assigned to him/her and an exercise for maintaining his/her good health. Details of the schedule management system 1 and the like will be described hereinafter.

As shown in FIG. 1, the schedule management system 1 includes a schedule management apparatus 10, a user terminal 20, a database 40, and a network 50. The schedule management apparatus 10 can also be referred to as a schedule management system. The schedule management apparatus 10, the user terminal 20, and the database 40 are configured so that they can communicate with each other through a wired or wireless network 50.

The user terminal 20 is a terminal capable of performing communication, owned by or temporarily assigned to a user U1, such as a PC (Personal Computer) terminal, a mobile terminal such as a smartphone or a tablet-type terminal, or a dedicated communication terminal prepared for the schedule management system.

For example, the user U1 enters information about a target exercise amount of the user U1 for each predetermined period to the user terminal 20 by operating an operation screen, a keyboard, or the like of the user terminal 20. The user terminal 20 receives the information about the target exercise amount of the user U1 for each predetermined period, entered by the operation performed by the user U1, and transmits the received information to the schedule management apparatus through the network 50.

Note that the information about the target exercise amount of the user U1 for each predetermined period is information about an exercise amount that the user U1 wants to achieve for each predetermined period. Note that the predetermined period can be arbitrarily set and is, for example, one week, one month, or three months. For example, the target exercise amount of the user U1 for each predetermined period is expressed by a target calorie consumption of the user U1 for each predetermined period, but is not limited to this example. That is, it is expressed by a target amount of decrease in weight (a difference between a current body weight and a target body weight), a target amount of decrease in body fat percentage (a difference between a current body fat percentage and a target body fat percentage), or a target degree of change in body shape (a degree of change in the appearance of his/her body or the like). Alternatively, the user terminal 20, the schedule management apparatus 10, or the like may derive (e.g., calculate) a target calorie consumption from a target amount of decrease in weight, a target amount of decrease in body fat percentage, or a target degree of change in body shape, or the like. Note that it is preferred to further refer to information about at least one of the age, gender, height, and weight of the user U1 when the target calorie consumption is derived. In this way, it is possible to derive the target calorie consumption more reliably.

Note that the user terminal 20 may receive not only the information about the target exercise amount of the user U1 for each predetermined period, but also information about a working place (a first working place) where the user U1 should stay for a part of the predetermined period (a first period) and/or information about a working place (a second working place) to which the user U1 has determined that he/she cannot move, and transmit these received information items to the schedule management apparatus 10 through the network 50. Further, the user terminal 20 may also receive information about at least one of the above-described age, gender, height, and weight of the user U1, and transmit the received information to the schedule management apparatus 10 through the network 50.

Note that the information about the first working place where the user U1 should stay for the first period included in the predetermined period is information about, for example, a place where a meeting the user U1 himself/herself has to participate in is held or a place where an inventory task the user U1 himself/herself has to perform is carried out. Further, the information about the second working place to which the user U1 has determined that he/she cannot move is information about, for example, a working place located inside a building that the user U1 does not have a right to enter.

Further, although an example in which the user terminal 20 receives the information about the target exercise amount of the user U1 for each predetermined period and transmits the received information to the schedule management apparatus 10 through the network 50 is described in this embodiment, the present disclosure is not limited to this example. For example, a communication terminal other than the user terminal 20 may receive the information about the target exercise amount of the user U1 for each predetermined period and transmit the received information to the schedule management apparatus 10 through the network 50.

The schedule management apparatus 10 extracts, from the database 40, at least one working place with which the user U1 achieves an exercise amount corresponding to his/her target exercise amount for each predetermined period, and assigns the extracted at least one working place to the user U1 as a working place for the predetermined period (for each predetermined period, i.e., for a respective predetermined period).

Note that in the database 40, information about, for each of a plurality of predetermined working places, an exercise amount (e.g., a calorie consumption) that is achieved when the user U1 moves from his/her residence place to that working place is registered in advance. Note that such information about an exercise amount does not necessarily have be registered in itself in the database 40. That is, information necessary, by the place assignment unit 12 or the like, to derive (e.g., calculate) an exercise amount may be registered in the database 40.

Note that the exercise amount for each of the plurality of predetermined working places that is achieved as the user U1 moves from his/her residence place to the working place may include an exercise amount during a work performed in the working place. The exercise amount during the work at the working place may also include, for example, an exercise amount that is achieved when the user U1 works while sitting on a balance ball or while using a foot pedaling machine.

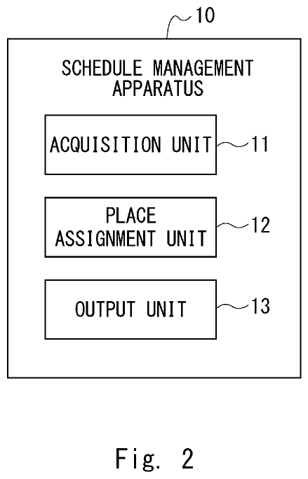

FIG. 2 is a block diagram showing an example of a configuration of the schedule management apparatus 10. As shown in FIG. 2, the schedule management apparatus 10 includes at least an acquisition unit 11, a place assignment unit 12, and an output unit 13.

The acquisition unit 11 acquires information about the target exercise amount of the user U1 for each predetermined period, transmitted from the user terminal 20 or the like.

The place assignment unit 12 extracts, from the database 40, at least one working place with which the user U1 achieves an exercise amount corresponding to his/her target exercise amount for each predetermined period (i.e., for a respective predetermined period), and assigns the extracted at least one working place to the user U1 as a working place for the predetermined period.

The output unit 13 outputs information about the working place assigned to the user U1. The information output from the output unit 13 is, for example, transferred to the user terminal 20 through the network 50 and displayed on a monitor of the user terminal 20. In this way, the user U1 can know the working place assigned to himself/herself.

Note that when the schedule management apparatus 10 acquires information about a first working place where the user U1 should stay for a first period included in the predetermined period, it extracts, from the database 40, at least one working place which includes the first working place for the first period and with which the user U1 achieves an exercise amount corresponding to his/her target exercise amount for each predetermined period, and assigns the extracted at least one working place to the user U1 as the working place for the predetermined period. Further, when the schedule management apparatus 10 acquires information about a second working place to which the user U1 has determined that he/she cannot move, it extracts, from the database 40, at least one working place which is different from the second working place and with which the user U1 achieves an exercise amount corresponding to his/her target exercise amount for each predetermined period, and assigns the extracted at least one working place to the user U1 as the working place for the predetermined period.

As described above, the schedule management system 1 according to this embodiment extracts, from the database 40, at least one working place with which the user U1 achieves an exercise amount corresponding to his/her target exercise amount for each predetermined period, and assigns the extracted at least one working place to the user U1 as a working place for the predetermined period. In this way, the schedule management system 1 according to this embodiment can assign, to the user U1, a working place with which the user U1 can achieve an appropriate exercise amount. That is, the schedule management system 1 according to this embodiment can propose a work plan by which the user U1 achieves both the performance of a task assigned to him/her and an exercise for maintaining his/her good health.

Note that in the schedule management apparatus 10, the acquisition unit 11 may acquire information about a congestion state of each of the plurality of predetermined working places. In this case, the place assignment unit 12 assigns a working place to the user U1 based on the congestion state of each of the plurality of predetermined working places. For example, when a workplace A1 among the plurality of predetermined working places is crowded, the place assignment unit 12 assigns a working place other than the workplace A1 to the user U1.

Further, in the schedule management apparatus 10, the acquisition unit 11 may further acquire, as feedback information, information about an actual exercise amount performed by the user U1 as he/she moves from his/her residence place to the predetermined working place. For example, the acquisition unit 11 acquires, as feedback information, information about a heart rate, a pulse rate, the number of steps, or a calorie consumption of the user U1 measured by a wearable terminal (not shown) attached to the user U1, and/or acquires, as feedback information, information about an amount of change in the weight of the user U1 measured by a scale (not shown). Then, the acquired feedback information is registered in the database 40. That is, the information registered in the database 40 is updated by the feedback information. After that, the place assignment unit 12 extracts, from the database 40, which has been updated by the feedback information, at least one working place with which the user U1 achieves an exercise amount corresponding to his/her target exercise amount for each predetermined period. By doing so, the place assignment unit 12 can extract at least one working place with which the user U1 achieves an exercise amount corresponding to his/her target exercise amount for each predetermined period more accurately.

### (Operation of Schedule Management System 1)

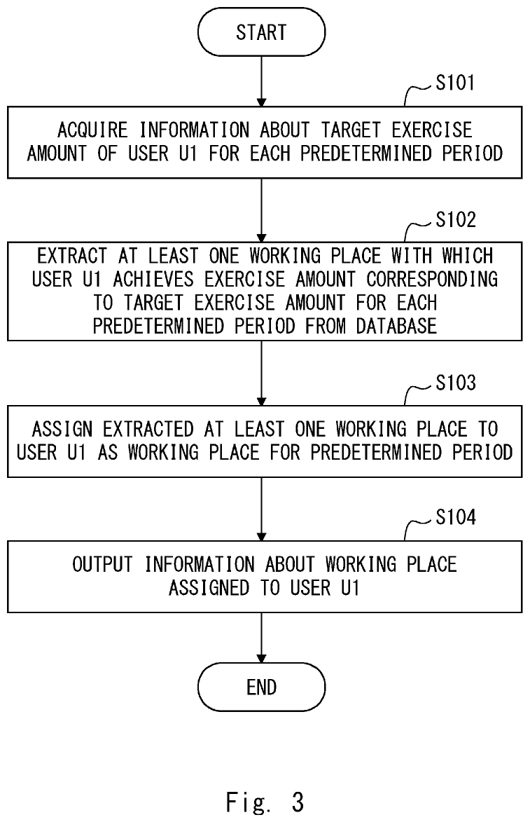

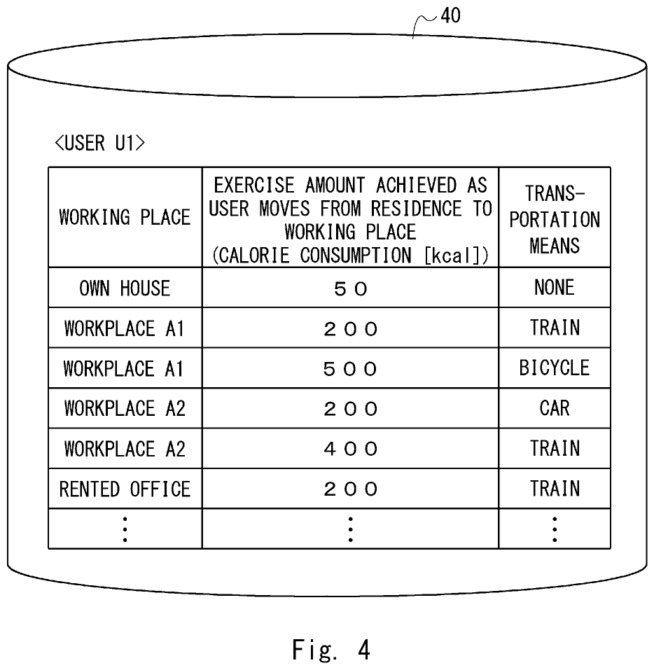

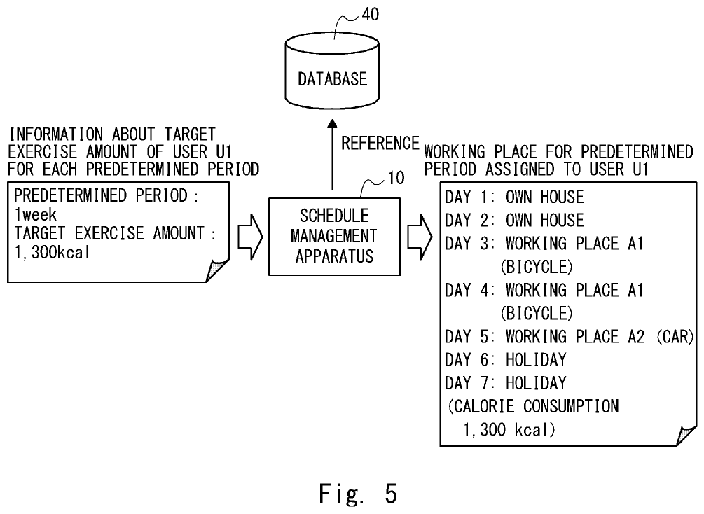

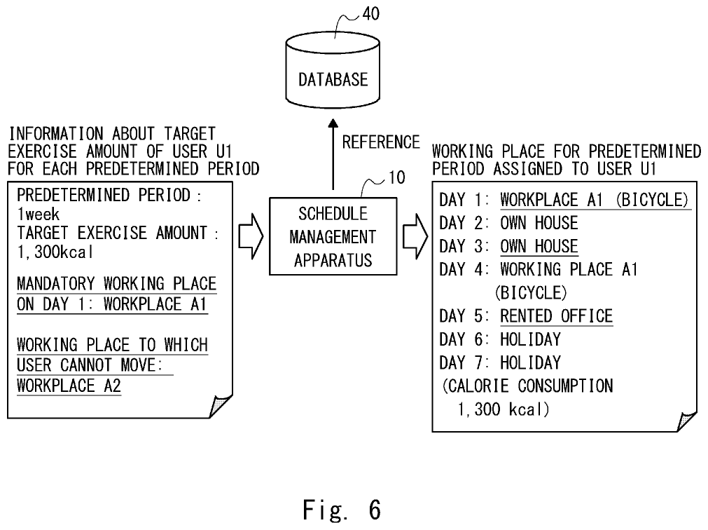

Next, operations performed by the schedule management system 1 will be described with reference to FIGS. 3 to 6. FIG. 3 is a flowchart showing operations performed by the schedule management system 1. FIG. 4 is a conceptual diagram showing an example of, for each of a plurality of predetermined working places, an exercise amount that is achieved as a user U1 moves from his/her residence place to that working place, registered in the database 40. FIG. 5 is a conceptual diagram for explaining an example of operations performed by the schedule management system 1. FIG. 6 is a conceptual diagram for explaining another example of operations performed by the schedule management system 1. Note that in the examples shown in FIGS. 4 to 6, calorie consumptions are used as examples of the exercise amount.

As shown in FIG. 4, in the database 40, information about, for each of a plurality of predetermined working places, calories consumed by the user U1 (i.e., a calorie consumption of the user U1) as he/she moves from his/her residence place to that working place is registered. Specifically, in the database 40, the following information is registered: when the working place is in his/her own house, the calorie consumption is 50 kcal; when the working place is a workplace A1 and the transportation means is a train, the calorie consumption is 200 kcal; when the working place is the workplace A1 and the transportation means is a bicycle, the calorie consumption is 500 kcal; when the working place is a workplace A2 and the transportation means is a car, the calorie consumption is 200 kcal; when the working place is the workplace A2 and the transportation means is a train, the calorie consumption is 400 kcal; and when the working place is a predetermined rented office and the transportation means is a train, the calorie consumption is 200 kcal.

Firstly, the user terminal 20 receives information about a target exercise amount of the user U1 for each predetermined period, which has been entered by the operation performed by the user U1, and transmits the received information to the schedule management apparatus 10 through the network 50.

In the example shown in FIG. 5, the predetermined period is set to one week, and a target calorie consumption, which is the target exercise amount of the user U1 for each week, is set to 1,300 kcal.

In the schedule management apparatus 10, the acquisition unit 11 acquires the information about the target exercise amount of the user U1 for each predetermined period, transmitted from the user terminal 20 or the like (Step S101).

After that, in the schedule management apparatus 10, the place assignment unit 12 extracts at least one working place with which the user U1 achieves an exercise amount corresponding to his/her target exercise amount for each predetermined period from the database 40 (Step S102). Then, the place assignment unit 12 assigns the extracted at least one working place to the user U1 as a working place for the predetermined period (Step S103).

In the example shown in FIG. 5, the schedule management apparatus 10 assigns, as working places, his/her own house on Day 1, his/her own house on Day 2, a workplace A1 (the transportation means is a bicycle) on Day 3, the workplace A1 (the transportation means is the bicycle) on Day 4, and a workplace A2 (the transportation means is a car) on Day 5 to the user U1 so that the calorie consumption of the user U1 for each week becomes equal to or close to the target calorie consumption. Note that since Days 6 and 7 are holidays, no working place is assigned on these days. Note that as shown in the database 40 shown in FIG. 4, the total calorie consumption by the traveling from the residence place of the user U1 to the working place is 1,300 kcal, and is equal to the target calorie consumption of the user U1.

After that, the output unit 13 outputs information about the working places assigned to the user U1 (Step S104). The information output from the output unit 13 is, for example, transferred to the user terminal 20 through the network 50 and displayed on the monitor of the user terminal 20. In this way, the user U1 can know the working places assigned to him/her.

Note that as already described above, the user terminal 20 may receive not only the information about the target exercise amount of the user U1 for each predetermined period, but also information about a first working place where the user U1 should stay for a part of the predetermined period (a first period) and/or information about a second working place to which the user U1 has determined that he/she cannot move, and transmit these received information items to the schedule management apparatus 10 through the network 50. This feature will be briefly described hereinafter with reference to FIG. 6.

In the example shown in FIG. 6, the target calorie consumption, which is the target exercise amount of the user U1 for each week, is set to 1,300 kcal. Further, a building A1 is set as a mandatory working place for the user U1 on Day 1. Further, a building A2 is set as a working place to which the user U1 cannot move. In this case, in consideration of the set conditions, the schedule management apparatus 10 assigns, as working places, the workplace A1 (the transportation means is a bicycle) on Day 1, his/her own house on Day 2, his/her own house on Day 3, the workplace A1 (the transportation means is the bicycle) on Day 4, and the rented office on Day 5 to the user U1 so that the calorie consumption of the user U1 for each week becomes equal to or close to the target calorie consumption. That is, the schedule management apparatus 10 assigns, to the user U1, the workplace A1 instead of his/her own house as the working place on Day 1, his/her own house instead of the workplace A1 as the working place on Day 3, and the rented office instead of the workplace A2 as the working place on Day 5. Note that as shown in the database 40 shown in FIG. 4, the total calorie consumption by the traveling from the residence place of the user U1 to the working place is 1,300 kcal, and is equal to the target calorie consumption of the user U1.

As described above, the schedule management system 1 according to this embodiment extracts, from the database 40, at least one working place with which the user U1 achieves an exercise amount corresponding to his/her target exercise amount for each predetermined period, and assigns the extracted at least one working place to the user U1 as a working place for the predetermined period. In this way, the schedule management system 1 according to this embodiment can assign, to the user U1, a working place with which the user U1 can achieve an appropriate exercise amount. That is, the schedule management system 1 according to this embodiment can propose a work plan by which the user U1 achieves both the performance of a task assigned to him/her and an exercise for maintaining his/her good health.

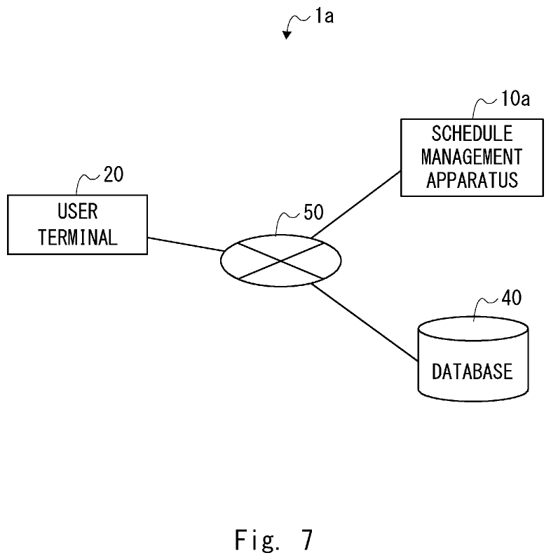

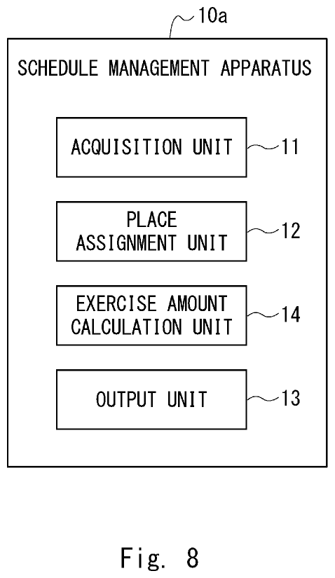

Note that although an example in which information about, for each of a plurality of predetermined working places, an exercise amount that is achieved when the user U1 moves from his/her residence place to that working place is registered in advance in the database 40 has been described in this embodiment, the present disclosure is not limited to such examples. The information about, for each of a plurality of predetermined working places, an exercise amount that is achieved when the user U1 moves from his/her residence place to that working place may be calculated by the schedule management apparatus 10 and then registered in the database 40. This feature will be briefly described hereinafter with reference to FIGS. 7 and 8.

### Modified Example of Schedule Management System 1

FIG. 7 is a block diagram showing a modified example of the schedule management system 1 as a schedule management system 1a. The schedule management system 1a includes a schedule management apparatus 10a in place of the schedule management apparatus 10. FIG. 8 is a block diagram showing an example of a specific configuration of the schedule management apparatus 10a. As compared with the schedule management apparatus 10, the schedule management apparatus 10a further includes an exercise amount calculation unit 14.

For example, the user U1 also enters information about his/her residence place and a plurality of predetermined working places to the user terminal 20 by operating an operation screen, a keyboard, or the like of the user terminal 20. The user terminal 20 receives the information about the residence place of the user U1 and the plurality of predetermined working places entered by the operation performed by the user U1, and transmits the received information to the schedule management apparatus 10 through the network 50.

Note that the information about the residence place of the user U1 is, for example, information about a place where the residence place of the user U1 is located. Further, the information about the predetermined working place is information about a place where the user U1 performs a task assigned to him/her and/or information about a work environment. Examples of predetermined working places include the own house (the residence place) of the user U1, a working space in his/her company or the like, and a rented office where the user U1 is permitted to work.

Note that although an example in which the user terminal 20 receives information about the residence place of the user U1 and a plurality of predetermined working places, and transmits the received information to the schedule management apparatus 10 through the network 50 is described in this embodiment, the present disclosure is not limited to this example. For example, a communication terminal other than the user terminal 20 may receive information about the residence place of the user U1 and a plurality of predetermined working places, and transmit the received information to the schedule management apparatus 10 through the network 50.

In the schedule management apparatus 10a, the acquisition unit 11 acquires the information about the residence place of the user U1 and the plurality of predetermined working places, transmitted from the user terminal 20 or the like. The exercise amount calculation unit 14 calculates, based on the information about the residence place of the user U1 and the plurality of predetermined working places acquired by the acquisition unit 11, for each of the plurality of predetermined working places, an exercise amount that is achieved when the user U1 moves from his/her residence place to that working place, and registers the calculated exercise amounts in the database 40.

Note that the exercise amount calculation unit 14 preferably calculates the exercise amount while taking the traveling environment such as the transportation means and the traveling route into consideration. For example, the exercise amount calculation unit 14 performs the calculation in such a manner that the longer the traveling time, the larger the exercise amount becomes, and the shorter the traveling time, the smaller the exercise amount becomes. Alternatively, the exercise amount calculation unit 14 performs the calculation in such a manner that when the transportation means is walking or a bicycle, the exercise amount becomes larger than when the transportation means is a train or a car. Further, the exercise amount calculation unit 14 may calculate, for each of the plurality of predetermined working places, an exercise amount that is achieved when the user U1 moves from his/her residence place to that working place while incorporating an exercise amount that is achieved during the work performed in the working place thereinto. Note that the exercise amount achieved during the work in the working place may also include, for example, an exercise amount that is achieved when the user U1 works while sitting on a balance ball or while using a foot pedaling machine.

The rest of the configuration of the schedule management system 1a is similar to that of the schedule management system 1, and therefore the description thereof is omitted.

### Second Embodiment

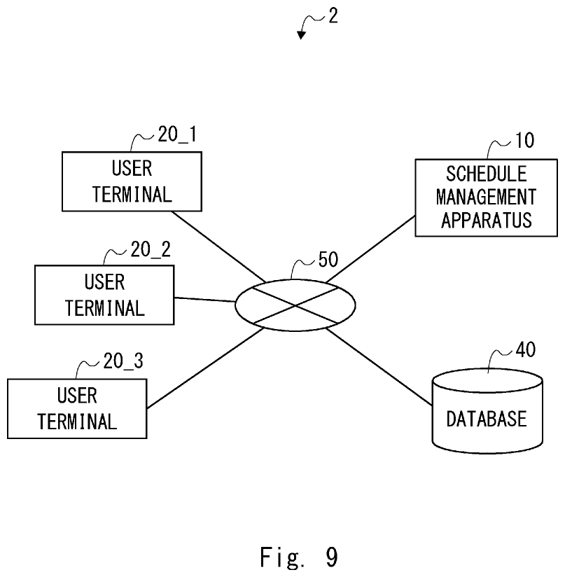

FIG. 9 is a block diagram showing an example of a configuration of a schedule management system 2 according to a second embodiment. The schedule management system 2 according to this embodiment assigns a working place for each predetermined period not only to the user U1, but also to each of users other than the user U1.

As shown in FIG. 9, the schedule management system 2 includes a schedule management apparatus 10, three user terminals 20, a database 40, and a network 50. Hereafter, the three user terminals 20 are referred to as user terminals 20_1, 20_2 and 20_3, respectively. Note that although three user terminals 20 are provided in the example shown in FIG. 9, the number of user terminals is not limited to three. That is, the number of user terminals 20 provided in the schedule management system may be any number larger than one.

The user terminals 20_1 to 20_3 are terminals capable of performing communication, owned by or temporarily assigned to users U1 to U3, respectively. The user terminal 20_1 receives information about a target exercise amount of the user U1 for each predetermined period, entered by an operation performed by the user U1, and transmits the received information to the schedule management apparatus 10 through the network 50. The user terminal 20_2 receives information about a target exercise amount of the user U2 for each predetermined period, entered by an operation performed by the user U2, and transmits the received information to the schedule management apparatus 10 through the network 50. The user terminal 20_3 receives information about a target exercise amount of the user U3 for each predetermined period, entered by an operation performed by the user U3, and transmits the received information to the schedule management apparatus 10 through the network 50.

The schedule management apparatus 10 extracts, from the database 40, for each of the users U1 to U3, at least one working place with which that user achieves an exercise amount corresponding to his/her target exercise amount for each predetermined period, and assigns, to each of the users U1 to U3, the extracted at least one working place as a working place for the predetermined period.

More specifically, in the schedule management apparatus 10, the acquisition unit 11 acquires information about the target exercise amount for each predetermined period for each of the users U1 to U3, transmitted from a respective one of the user terminals 20_1 to 20_3.

After that, in the schedule management apparatus 10, the place assignment unit 12 extracts, from the database 40, for each of the users U1 to U3, at least one working place with which that user achieves an exercise amount corresponding to his/her target exercise amount for each predetermined period, and assigns, to each of the users U1 to U3, the extracted at least one working place as a working place for the predetermined period.

Note that when the users U1 to U3 belong to the same group G1, the place assignment unit 12 may assign a working place to each of the users U1 to U3 while dividing a plurality of tasks assigned to this group G1 among the users U1 to U3. The group G1 is, for example, a department or the like of a company or the like to which the users U1 to U3 belong. Note that the place assignment unit 12 may assign all of the plurality of tasks assigned to the group G1 to the users U1 to U3 while dividing the plurality of tasks among the users U1 to U3. Further, when the sum total of exercise amounts of the plurality of tasks assigned to the group G1 is smaller than the sum total of the target exercise amounts of the users U1 to U3, the place assignment unit 12 may extract, for each of the users U1 to U3, at least one task with which that user achieves an exercise amount corresponding to the ratio of his/her target exercise amount to the sum total of the target exercise amounts of the users U1 to U3, and assign, for each of the users U1 to U3, the extracted at least one task to that user as a task for each predetermined period.

The output unit 13 outputs information about the working places assigned to the users U1 to U3, respectively. The information output from the output unit 13 is, for example, transferred to the user terminals 20_1 to 20_3 through the network 50 and displayed on monitors thereof. In this way, each of the users U1 to U3 can know the working place assigned to him/her.

### Example of Operation of Schedule Management System 2

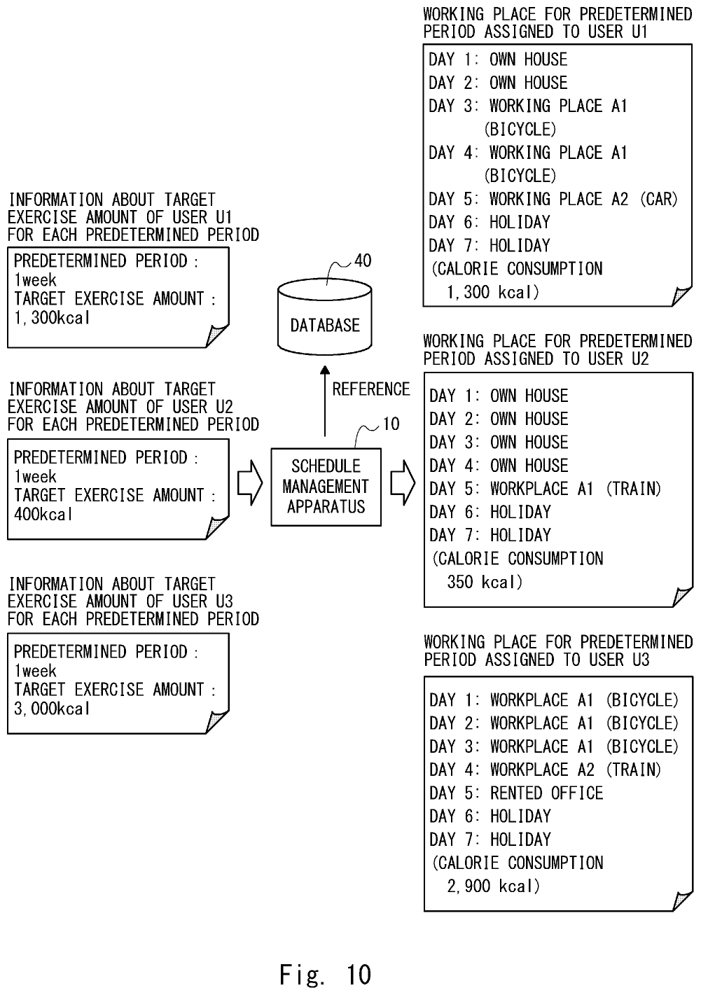

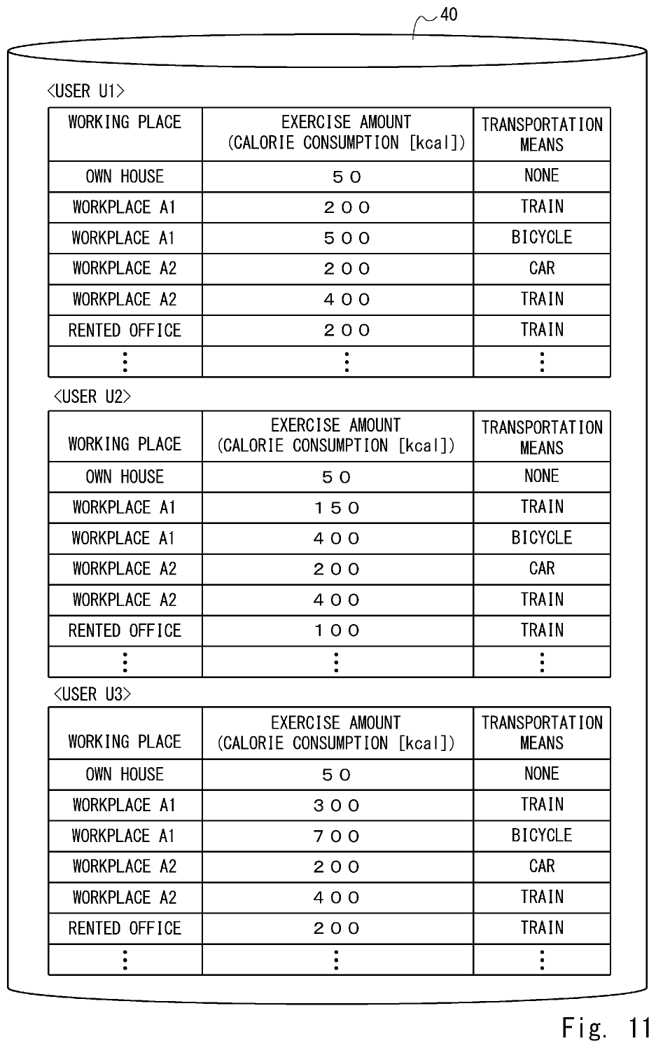

FIG. 10 is a conceptual diagram for explaining an example of operations performed by the schedule management system 2. Further, FIG. 11 is a conceptual diagram showing an example of, for each of the users U1 to U3 and for each of a plurality of predetermined working places, an exercise amount that is achieved as that user moves from his/her residence place to that working place, registered in the database 40. In the examples shown in FIGS. 10 and 11, calorie consumptions are used as examples of exercise amounts.

Note that as shown in FIG. 11, information about, for each of the users U1 to U3 and for each of a plurality of predetermined working places, calories consumed by that user (i.e., a calorie consumption of that user) as he/she moves from his/her residence place to the working place is registered in the database 40. Firstly, in the database 40, as an example of calories that are consumed when the user U1 moves from his/her residence place to each of a plurality of predetermined working places, the following information is registered: when the working place is in his/her own house, the calorie consumption is 50 kcal; when the working place is a workplace A1 and the transportation means is a train, the calorie consumption is 200 kcal; when the working place is the workplace A1 and the transportation means is a bicycle, the calorie consumption is 500 kcal; when the working place is a workplace A2 and the transportation means is a car, the calorie consumption is 200 kcal; when the working place is the workplace A2 and the transportation means is a train, the calorie consumption is 400 kcal; and when the working place is a predetermined rented office and the transportation means is a train, the calorie consumption is 200 kcal. Further, in the database 40, as an example of calories that are consumed when the user U2 moves from his/her residence place to each of a plurality of predetermined working places, the following information is registered: when the working place is in his/her own house, the calorie consumption is 50 kcal; when the working place is a workplace A1 and the transportation means is a train, the calorie consumption is 150 kcal; when the working place is the workplace A1 and the transportation means is a bicycle, the calorie consumption is 400 kcal; when the working place is a workplace A2 and the transportation means is a car, the calorie consumption is 200 kcal; when the working place is the workplace A2 and the transportation means is a train, the calorie consumption is 400 kcal; and when the working place is a predetermined rented office and the transportation means is a train, the calorie consumption is 100 kcal. Further, in the database 40, as an example of calories that are consumed when the user U3 moves from his/her residence place to each of a plurality of predetermined working places, the following information is registered: when the working place is in his/her own house, the calorie consumption is 50 kcal; when the working place is a workplace A1 and the transportation means is a train, the calorie consumption is 300 kcal; when the working place is the workplace A1 and the transportation means is a bicycle, the calorie consumption is 700 kcal; when the working place is a workplace A2 and the transportation means is a car, the calorie consumption is 200 kcal; when the working place is the workplace A2 and the transportation means is a train, the calorie consumption is 400 kcal; and when the working place is a predetermined rented office and the transportation means is a train, the calorie consumption is 200 kcal.

In the example shown in FIG. 10, the predetermined period is set to one week for all the users. Further, the target calorie consumption of the user U1, which is the target exercise amount of the user U1 for each week, is set to 1,300 kcal. The target calorie consumption of the user U2, which is the target exercise amount of the user U2 for each week, is set to 400 kcal. The target calorie consumption of the user U3, which is the target exercise amount of the user U3 for each week, is set to 3,000 kcal.

Note that the schedule management apparatus 10 assigns, as working places, his/her own house on Day 1, his/her own house on Day 2, a workplace A1 (the transportation means is a bicycle) on Day 3, the workplace A1 (the transportation means is the bicycle) on Day 4, and a workplace A2 (the transportation means is a car) on Day 5 to the user U1 so that the calorie consumption of the user U1 for each week becomes equal to or close to the target calorie consumption. Note that since Days 6 and 7 are holidays, no working place is assigned on these days. Note that as shown in the database 40 shown in FIG. 11, the total calorie consumption by the traveling from the residence place of the user U1 to the working place is 1,300 kcal, and is equal to the target calorie consumption of the user U1.

Further, the schedule management apparatus 10 assigns, as working places, his/her own house on Day 1, his/her own house on Day 2, his/her own house on Day 3, his/her own house on Day 4, and the workplace A1 (the transportation means is a train) on Day 5 to the user U2 so that the calorie consumption of the user U2 for each week becomes equal to or close to the target calorie consumption. Note that since Days 6 and 7 are holidays, no working place is assigned on these days. Note that as shown in the database 40 shown in FIG. 11, the total calorie consumption by the traveling by from the residence place of the user U2 to the working place is 350 kcal, and is close to the target calorie consumption of the user U2, which is 400 kcal.

Further, the schedule management apparatus 10 assigns, as working places, the workplace A1 (the transportation means is a bicycle) on Day 1, the workplace A1 (the transportation means is the bicycle) on Day 2, the workplace A1 (the transportation means is the bicycle) on Day 3, the workplace A2 (the transportation means is a train) on Day 4, and a rented office on Day 5 to the user U3 so that the calorie consumption of the user U3 for each week becomes equal to or close to the target calorie consumption. Note that since Days 6 and 7 are holidays, no working place is assigned on these days. Note that as shown in the database 40 shown in FIG. 11, the total calorie consumption by the traveling by from the residence place of the user U3 to the working place is 2,900 kcal, and is close to the target calorie consumption of the user U3, which is 3,000 kcal.

### Other Example of Operation of Schedule Management System 2

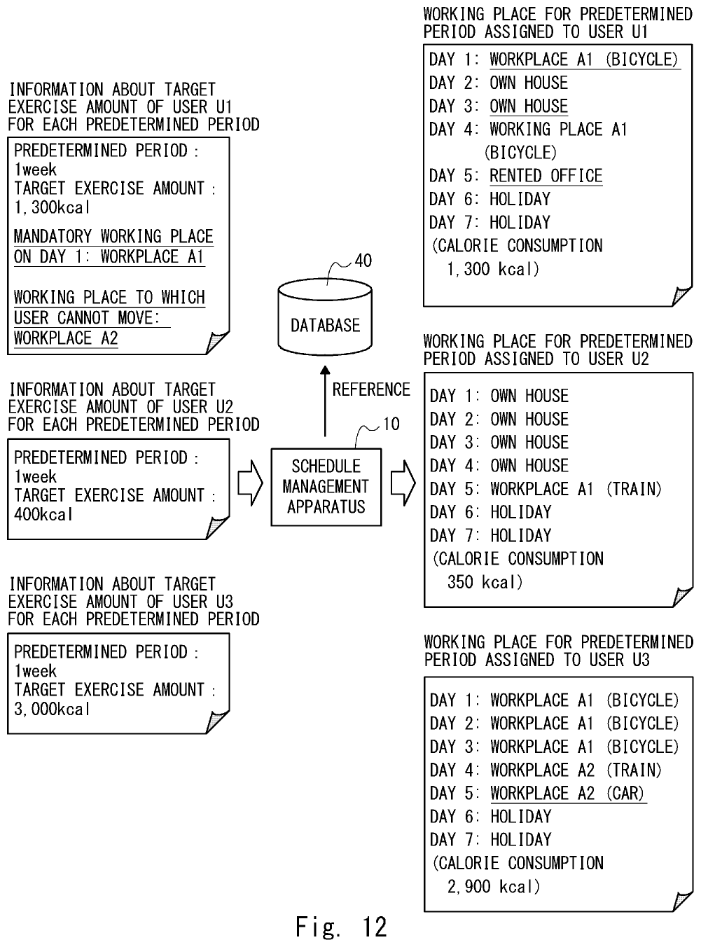

FIG. 12 is a conceptual diagram for explaining another example of operations performed by the schedule management system 2. In the example shown in FIG. 12, a target calorie consumption, which is the target exercise amount for each week for user U1, is set to 1,300 kcal. Further, a building A1 is set as a mandatory working place for the user U1 on Day 1. Further, a building A2 is set as a working place to which the user U1 cannot move.

In this case, in consideration of the set conditions, the schedule management apparatus 10 assigns, as working places, the workplace A1 (the transportation means is a bicycle) on Day 1, his/her own house on Day 2, his/her own house on Day 3, the workplace A1 (the transportation means is the bicycle) on Day 4, and the rented office on Day 5 to the user U1 so that the calorie consumption of the user U1 for each week becomes equal to or close to the target calorie consumption. That is, the schedule management apparatus 10 assigns, to the user U1, the workplace A1 instead of his/her own house as the working place on Day 1, his/her own house instead of the workplace A1 as the working place on Day 3, and the rented office instead of the workplace A2 as the working place on Day 5. Note that as shown in the database 40 shown in FIG. 11, the total calorie consumption by the traveling from the residence place of the user U1 to the working place is 1,300 kcal, and is equal to the target calorie consumption of the user U1.

Further, the schedule management apparatus 10 assigns, as working places, the workplace A1 (the transportation means is a bicycle) on Day 1, the workplace A1 (the transportation means is the bicycle) on Day 2, the workplace A1 (the transportation means is the bicycle) on Day 3, the workplace A2 (the transportation means is a train) on Day 4, and the workplace A2 (the transportation means is a car) on Day 5 to the user U3 so that the calorie consumption of the user U3 for each week becomes equal to or close to the target calorie consumption. That is, the schedule management apparatus 10 assigns the workplace A2 instead of the rented office as the working place on Day 5 to the user U3. That is, the working places of the users U1 and U3 on Day 5 are interchanged. Note that as shown in the database 40 shown in FIG. 11, the total calorie consumption by the traveling by from the residence place of the user U3 to the working place is 2,900 kcal, and is close to the target calorie consumption of the user U3, which is 3,000 kcal.

The working places assigned to the user U2 are similar to those in the example shown in FIG. 10, and therefore descriptions thereof are omitted.

As described above, the schedule management system 2 according to this embodiment extracts, from the database 40, for each of the users U1 to U3, at least one working place with which that user achieves an exercise amount corresponding to his/her target exercise amount for each predetermined period, and assigns the extracted at least one working place as a working place for the predetermined period. In this way, the schedule management system 2 according to this embodiment can assign, to each of the users U1 to U3, working places by which that user achieves an appropriate exercise amount. That is, the schedule management system 2 according to this embodiment can propose, for each of the users U1 to U3, a work plan by which that user can achieve both the performance of a task assigned to him/her and an exercise for maintaining his/her good health.

Note that the present disclosure is not limited to the above-described embodiments, and they can be modified as appropriate without departing from the scope and spirit of the disclosure.

Further, in the present disclosure, some or all of the control processes performed in the schedule management system can be implemented by having a CPU (Central Processing Unit) execute a computer program.

In the aforementioned program includes instructions (or software codes) that, when loaded into a computer, cause the computer to perform one or more of the functions described in the embodiments. The program may be stored in a non-transitory computer readable medium or a tangible storage medium. By way of example, and not a limitation, the non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A schedule management system comprising:
an acquisition unit configured to acquire information about a target exercise amount of a first user for each predetermined period;
a place assignment unit configured to extract at least one working place from a database and assign the extracted at least one working place to the first user as a working place for the predetermined period, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered; and
an output unit configured to output information about the working place assigned to the first user by the place assignment unit.

2. The schedule management system according to claim 1, wherein the exercise amount for each of the plurality of predetermined working places that is achieved when the first user moves from the residence place of the first user to the working place is determined based on a traveling environment from the residence place to the working place.

3. The schedule management system according to claim 1, further comprising an exercise amount calculation unit, wherein
the acquisition unit further acquires information about the residence place of the first user and the plurality of predetermined working places, and
the exercise amount calculation unit calculates, for each of the plurality of predetermined working places, an exercise amount that is achieved when the first user moves from the residence place of the first user to that working place based on the information about the residence place and the plurality of predetermined working places acquired by the acquisition unit, and registers the calculated exercise amounts in the database.

4. The schedule management system according to claim 1, wherein the exercise amount for each of the plurality of predetermined working places that is achieved when the first user moves from the residence place to the working place includes an exercise amount during a work performed in the working place.

5. The schedule management system according to claim 1, wherein
the acquisition unit further acquires information about a first working place where the first user should stay for a first period included in the predetermined period, and
the place assignment unit extracts, from the database, at least one working place which includes the first working place for the first period and with which the first user achieves the exercise amount corresponding to his/her target exercise amount for each predetermined period, and assigns the extracted at least one working place to the first user as the working place for the predetermined period.

6. The schedule management system according to claim 1, wherein
the acquisition unit further acquires information about a second working place to which the first user has determined that he/she cannot move, and
the place assignment unit extracts, from the database, at least one working place which is different from the second working place and with which the first user achieves the exercise amount corresponding to his/her target exercise amount for each predetermined period, and assigns the extracted at least one working place to the first user as the working place for the predetermined period.

7. The schedule management system according to claim 1, wherein
the acquisition unit acquires information about a congestion state of each of the plurality of predetermined working places, and
the place assignment unit extracts, from the database and based on the congestion states, at least one working place with which the first user achieves the exercise amount corresponding to his/her target exercise amount for each predetermined period, and assigns the extracted at least one working place to the first user as the working place for the predetermined period.

8. The schedule management system according to claim 1, wherein the acquisition unit acquires, as feedback information, information about an actual exercise amount performed by the first user as he/she has moved to the working place assigned to the first user, and registers the acquired actual exercise amount in the database.

9. The schedule management system according to claim 8, wherein the information about the actual exercise amount performed by the first user is at least one of a heart rate, a pulse rate, the number of steps, a calorie consumption of the first user acquired by a wearable terminal attached to the first user, and an amount of change in a weight of the first user measured by a scale.

10. The schedule management system according to claim 1, wherein the target exercise amount is a target calorie consumption of the first user.

11. The schedule management system according to claim 1, wherein the target exercise amount is determined based on a difference between a target weight and a current weight of the first user.

12. The schedule management system according to claim 1, wherein the target exercise amount is determined based on a difference between a difference between a target body fat percentage and a current body fat percentage of the first user.

13. The schedule management system according to claim 1, wherein the target exercise amount is determined based on a difference between a target body shape and a current body shape of the first user.

14. The schedule management system according to claim 1, wherein the output unit displays information about the working place assigned to the first user on a monitor.

15. The schedule management system according to claim 1, wherein
the acquisition unit acquires, for each of a plurality of users including the first user, information about the target exercise amount for each predetermined period,
the place assignment unit extracts, from the database, for each of the plurality of users, at least one working place with which that user achieves an exercise amount corresponding to his/her target exercise amount for each predetermined period, and assigns the extracted at least one working place to that user as the working place for the predetermined period, and
the output unit outputs information about the working places assigned to the users by the place assignment unit.

16. A method for controlling a schedule management system, comprising:
acquiring information about a target exercise amount of a first user for each predetermined period;
extracting at least one working place from a database, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered;
assigning the at least one working place extracted from the database to the first user as a working place for the predetermined period; and
outputting information about the working place assigned to the first user.

17. A non-transitory computer readable storage medium storing a control program for causing a computer to perform:
a process for acquiring information about a target exercise amount of a first user for each predetermined period;
a process for extracting at least one working place from a database, the working place being a place with which the first user achieves an exercise amount corresponding to his/her target exercise amount for each predetermined period, and in the database, information about, for each of a plurality of predetermined working places, an exercise amount that is achieved when the first user moves from his/her residence place to that working place or information necessary to determine this exercise amount being registered;
a process for assigning the at least one working place extracted from the database to the first user as a working place for the predetermined period; and
a process for outputting information about the working place assigned to the first user.
